(19) United States
(12) Patent Application Publication
(10) Pub. No.: US 2003/0207941 A1
(43) Pub. Date: Nov. 6, 2003

(54) Method of treating vascular endothelial growth factor mediated vascular disorders

(76) Inventors: David P. Bingaman, Fort Worth, TX (US); Michael A. Kapin, Arlington, TX (US); Daniel A. Gamache, Arlington, TX (US); Gustav Graff, Cleburne, TX (US); John M. Yanni, Burleson, TX (US)

Correspondence Address: ALCON RESEARCH, LTD., R&D COUNSEL, Q-148, 6201 SOUTH FREEWAY, FORT WORTH, TX, 76134-2099, US

(21) Appl. No.: 10/417,466

(22) Filed: Apr. 16, 2003

Related U.S. Application Data
(60) Provisional application No. 60/377,429, filed on May 3, 2002.

(51) Int. Cl. A61K 31/195

(52) U.S. Cl. 514/567

(57) ABSTRACT

The use of amfenac and derivatives, including nepafenac, to treat vascular endothelial growth factor mediated vascular disorders.

This application claims priority from U.S. S. No. 60/377,429, filed May 3, 2002.

This invention relates to the use of 2-amino-3-benzoylbenzene acetic acid (amfenac) to treat or prevent vascular endothelial growth factor (VEGF) mediated vascular disorders.

### BACKGROUND OF THE INVENTION

It has been previously shown that certain nonsteroidal antiinflammatory drugs (NSAIDs) can inhibit the formation of new blood vessels (angiogenesis) in pathologic conditions, as well as vascular leakage in certain inflammation models. The ability of most NSAIDs to influence vascular permeability and angiogenesis appears to be associated with their ability to block the cyclo-oxygenase enzymes (COX-1 and -2). Blockade of COX-1 and -2 is associated with a decrease in inflammatory mediators, such as PGE₂. Moreover, it appears that PGE₂ inhibition results in decreased expression and production of vascular endothelial growth factor (VEGF). VEGF is known to produce vascular leakage and angiogenesis in the eye of preclinical models. Also, increased levels of VEGF have been found in neovascular tissues and extracellular fluid from the eyes of patients with diabetic retinopathy and age-related macular degeneration. Thus, NSAIDs may inhibit vascular leakage and angiogenesis by modulating PGE₂ levels and its effects on VEGF expression and activity. This theory is supported by work involving animal tumor models which demonstrate that systemic administration of COX-2 inhibitors decreases PGE₂ and VEGF tissue levels and thereby prevent tumor-induced angiogenesis. In these models, VEGF activity and angiogenesis are restored by adding exogenous PGE₂ during continued COX-2 blockade. However, NSAIDs appear to have variable activity in animal models of ocular neovascularization (NV), in that selective COX inhibitors do not appear to inhibit choroidal neovascularization. In fact, these studies have called into question the role of COX-1 and/or COX-2 in the development of CNV.

3-benzoylphenylacetic acid and certain of its derivatives are known to possess anti-inflammatory activity. U.S. Pat. Nos. 4,254,146, 4,045,576, 4,126,635, and 4,503,073, and U.K. Patent Application Nos. 2,071,086A and 2,093,027A disclose various 3-benzoylphenylacetic acids, salts and esters, and hydrates thereof, having anti-inflammatory activity. U.S. Pat. No. 4,568,695 discloses 2-amino-3-benzoylphenylethyl alcohols having anti-inflammatory activity. U.S. Pat. No. 4,313,949 discloses 2-amino-3-benzoyl-phenylacetamides having anti-inflammatory activity.

Certain derivatives of 2-amino-3-benzoylbenzeneacetic acid (amfenac) and 2-amino-3-(4-chloro-benzoyl)benzeneacetic acid have also been evaluated by Walsh et al., J. Med Chem., 33:2296-2304 (1990), in an attempt to discover nonsteroidal anti-inflammatory prodrugs with minimal or no gastrointestinal side effects upon oral administration.

U.S. Pat. No. 4,683,242 teaches the transdermal administration of 2-amino-3-benzoylphenylacetic acids, salts, and esters, and hydrates and alcoholates thereof to control inflammation and alleviate pain.

U.S. Pat. No. 4,910,225 teaches certain benzoylphenylacetic acids for local administration to control ophthalmic, nasal, or otic inflammation. Only acetic acids are disclosed in the '225 patent; no esters or amides are mentioned or taught as anti-inflammatory agents for local administration to the eyes, nose and ears.

U.S. Pat. No. 5,475,034 discloses topically administrable compositions containing certain amide and ester derivatives of 3-benzyolphenylacetic acid, including nepafenac, useful for treating ophthalmic inflammatory disorders and ocular pain. According to the '034 patent at Col. 15, lines 35-39, “[s]uch disorders include, but are not limited to uveitis scleritis, episcleritis, keratitis, surgically-induced inflammation and endophthalmitis.”

U.S. Pat. No. 6,066,671 discloses the topical use of certain amide and ester derivatives of 3-benzoylphenylacetic acid, including nepafenac, for treating GLCIA glaucoma.

In commonly owned U.S. application Ser. No. 09/929,381, it was found that certain 3-benzoylphenlacetic acids and derivatives are useful for treating angiogenesis-related disorders.

### DETAILED DESCRIPTION OF THE INVENTION

Posterior segment neovascularization (NV) is the vision-threatening pathology responsible for the two most common causes of acquired blindness in developed countries: exudative age-related macular degeneration (AMD) and proliferative diabetic retinopathy. Currently the only approved treatments for posterior segment NV that occurs in exudative AMD is laser photocoagulation or photodynamic therapy with Visudyne; both therapies involve occlusion of affected vasculature which results in localized laser-induced damage to the retina. Surgical interventions with vitrectomy and membrane removal are the only options currently available for patients with proliferative diabetic retinopathy. No strictly pharmacologic treatment has been approved for use against posterior segment NV.

In addition to changes in the retinal microvasculature induced by hyperglycemia in diabetic patients leading to macular edema, proliferation of neovascular membranes is also associated with vascular leakage and edema of the retina. Where edema involves the macula, visual acuity worsens. In diabetic retinopathy, macular edema is the major cause of vision loss. Like angiogenic disorders laser photocoagulation is used to stabilize or resolve the edematous condition. Unfortunately, laser photocoagulation is a cytodestructive procedure, that while preventing further edema to develop, will alter the visual field of the affected eye.

An effective pharmacologic therapy for posterior segment NV and edema would likely provide substantial efficacy to the patient, thereby avoiding invasive surgical or damaging laser procedures. Effective treatment of the NV would improve the patient's quality of life and productivity within society. Also, societal costs associated with providing assistance and health care to the blind could be dramatically reduced.

Amfenac is an NSAID that is known to potently inhibit the activity of COX-1 and COX-2 enzymes. Unexpectedly, amfenac was found to inhibit both VEGF-induced cell proliferation and capillary tube formation in a dose-response fashion using a bovine retinal microvascular endothelial cell assay. To our knowledge, this blockade on VEGF effects by NSAIDs that occurs independently of COX inhibition, i.e., the ability to block the proangiogenic signal normally elicited by VEGF, is unique with regard to amfenac versus other NSAIDs. This unique activity may help explain, in part, our previous findings that topical nepafenac (the prodrug of amfenac) inhibited choroidal NV in a mouse model, where topical VOLTAREN® and ACULAR® had no effect. If this novel antiangiogenic activity occurs in man, amfenac (and topical nepafenac) could be used to more effectively treat diseases that involve VEGF signaling and in disease states where other NSAIDs would likely be less effective. Ophthalmic disorders associated with upregulation of VEGF that are potential indications for amfenac (topical nepafenac) would include exudative age-related macular degeneration, proliferative diabetic retinopathy, retinal vein occlusion, proliferative vitreoretinopathy, neovascular glaucoma, corneal angiogenesis, retinal microvasculopathy and retinal (macular) edema. Again, because amfenac is the active metabolite of nepafenac, which has the ability to reach the posterior segment following topical corneal application in preclinical models, it is possible to treat these VEGF-mediated ocular disorders using topical ocular administration of nepafenac.

According to the present invention, a therapeutically effective amount of a nepafenac is administered topically to an eye whereas local or systemic administration of amfenac would be used to treat and/or prevent VEGF mediated vascular disorders.

The doses of amfenac or nepafenac used in the treatment or prevention of VEGF medicated vascular abnormalities will depend on the type of abnormality to be prevented or treated, the age and body weight of the patient, and the form of preparation/route of administration. Compositions intended for topical ophthalmic administration will typically contain nepafenac in an amount of from about 0.001 to about 4.0% (w/v), preferably from about 0.01 to about 0.5% (w/v), with 1-2 drops once to several times a day. Likewise, representative doses for other forms of preparations are approximately 1-100 mg of amfenac/day/adult for injections or local administration and approximately 10-1000 mg of amfenac/adult for oral preparations, each administered once to several times a day.

Additional therapeutic agents may be added to supplement the use of nepafenac or amfenac.

The following examples are presented to illustrate various aspects of the present invention, but are not intended to limit the scope of the invention in any respect. The percentages are expressed on a weight/volume basis.

### EXAMPLE 1

The following formulations are representative of the topical compositions useful in the present invention.

Formulation 1

| | |
|---|---|
| Nepafenac | 0.01-0.5% |
| Polysorbate 80 | 0.01% |
| Benzalkonium Chloride | 0.01% + 10% excess |
| Disodium EDTA | 0.1% |
| Monobasic Sodium Phosphate | 0.03% |
| Dibasic Sodium Phosphate | 0.1% |
| Sodium Chloride | q.s. 290-300 mOsm/Kg |
| pH adjustment with NaOH and/or HCl | pH 4.2-7.4 |
| Water | q.s. 100% |

Formulation 2

| | |
|---|---|
| Nepafenac | 0.01-0.5% |
| Hydroxypropyl Methylcellulose | 0.5% |
| Polysorbate 80 | 0.01% |
| Benzalkonium Chloride | 0.01% + 5% excess |
| Disodium EDTA | 0.01% |
| Dibasic Sodium Phosphate | 0.2% |
| Sodium Chloride | q.s. 290-300 mOsm/Kg |
| pH adjustment with NaOH and/or HCl | pH 4.2-7.4 |
| Water | q.s. 100% |

Formulation 3

| | |
|---|---|
| Nepafenac | 0.1 + 6% excess |
| Carbopol 974P | 0.08% |
| Tyloxapol | 0.01% |
| Glycerin | 2.4% |
| Disodium EDTA | 0.01% |
| Benzalkonium Chloride | 0.01% |
| pH adjustment with NaOH and/or HCl | pH 7.5 ± 0.2 |
| Water | q.s. 100% |

### EXAMPLE 2

### Effect of AL06295A (Amfenac) on BRMEC (Bovine Retinal Microvascular Endothelial Cell) Proliferation

VEGF-induced BRMEC proliferation was measured using a modified MTT assay, BRMEC were plated at 3×10³ onto a fibronectin/hyaluronic acid matrix in 96-well plates (Coming). Growth medium was added for two days, followed by serum free medium (SFM) overnight, then by test medium containing 0 or 25 ng/ml VEGF in 100 μl of SFM. After 24 hours at 37° C./5%CO₂, 25 μl of MTT (3-(4,5-dimethylthiazol-2-yl)-2,5-diphenyl tetrazolium bromide) was added to each well and incubated for 4 hours. 100 μl of lysis buffer (20%SDS in 50:50 DMF:H₂O+2.0% acetic acid and 0.05%HCl) was then added to each well, and the plates were incubated overnight at 37° C. and read (SPECTRAmax 190, Molecular Devices; Sunnyvale, Calif.) at 570 nm. For experiments utilizing AL06295, 25 ng/ml VEGF was combined with the compound at 0.1, 0.3, 1.0 or 3 μM.

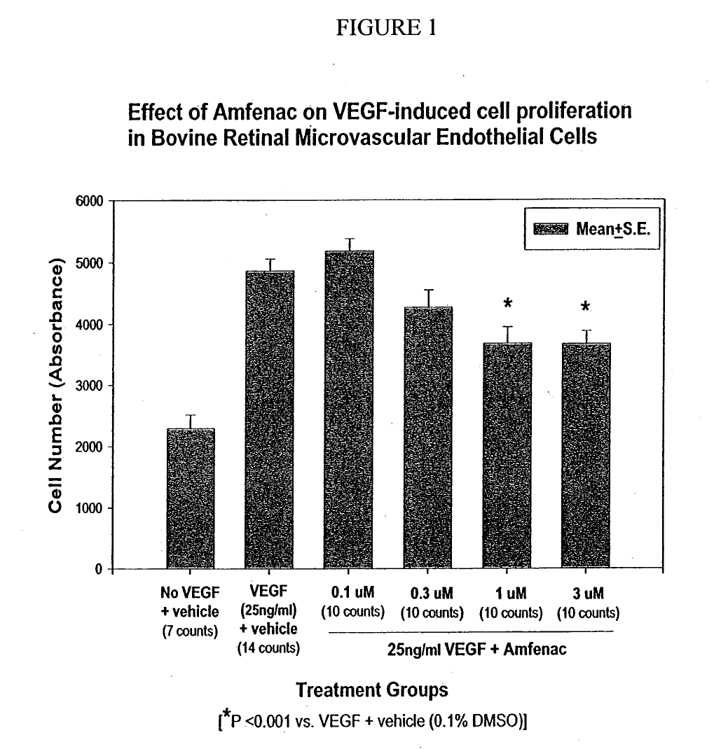

The results show that the 1 and 3 μM doses of amfenac significantly reduce VEGF induced BRMEC proliferation, see FIG. 1.

### EXAMPLE 3

### Effect of AL06295A (Amfenac) on BRMEC Tube Formation

A mixture of 8 vol of Vitrogen 100 (Cohesion; Palo Alto, Calif.), 1 vol. of 0.2N NaOH, and 1 vol. of 10× RPMI-1640 medium containing 5 μg/ml fibronectin and 5 μg/ml laminin was prepared and 400 μl was added to each well of a 24-well plate. After incubating for 3 hrs at 37° C. to solidify the gel, 10⁴ BRMEC were added to each well and incubated in growth medium for 3 days. Then the medium was carefully aspirated and 200 μl of the gel solution was layered on top of the cells and incubated at 37° C. for 1 hr. Following addition of growth medium for 24 hrs, 2 ml of test medium containing serum-free (SF) medium plus VEGF or SF medium plus VEGF and AL06295A were added to each well. The gels were assessed 24 hrs later.

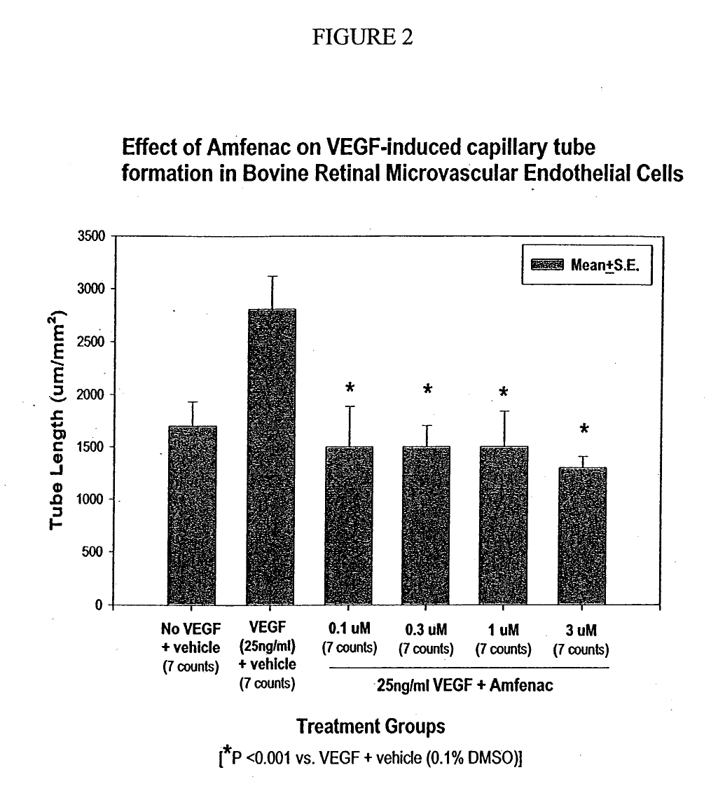

For quantitative analysis, six fields per treatment group were chosen from areas containing tubes; seven wells were used for each treatment. The lengths of the tubes were measured in digitized images, and the data are expressed in FIG. 2 as the total length per field of view in μm. The results show that all doses of amfenac significantly and potently inhibit VEGF induced capillary tube formation in BRMECs.

This invention has been described by reference to certain preferred embodiments; however, it should be understood that it may be embodied in other specific forms or variations thereof without departing from its special or essential characteristics. The embodiments described above are therefore considered to be illustrative in all respects and not restrictive, the scope of the invention being indicated by the appended claims rather than by the foregoing description.

We claim:

1. A method for treating vascular endothelial growth factor mediated vascular disorders, which comprises, administering a pharmaceutically effective amount of amfenac.

2. The method of claim 1 wherein the disorder is selected from the group consisting of exudative age related macular degeneration, proliferative diabetic retinopathy, retinal vein occlusion, proliferative vitreoretinopathy, neovascular glaucoma, corneal angiogenesis, retinal microvasculopathy, and retinal (macular) edema.

3. A method for treating vascular endothelial growth factor mediated vascular disorders, which comprises, administering a pharmaceutically effective amount of nepafenac.

4. The method of claim 3 wherein the disorder is selected from the group consisting of exudative age related macular degeneration, proliferative diabetic retinopathy, retinal vein occlusion, proliferative vitreoretinopathy, neovascular glaucoma, corneal angiogenesis, retinal microvasculopathy, and retinal (macular) edema.

5. The method of claim 1 wherein the disorder is an ophthalmic disorder.

6. The method of claim 3 wherein the disorder is an ophthalmic disorder.

7. The method of claim 2 wherein the disorder is retinal (macular) edema.

8. The method of claim 4 wherein the disorder is retinal (macular) edema.

9. The method of claim 2 wherein the disorder is proliferative diabetic retinopathy.

10. The method of claim 4 wherein the disorder is proliferative diabetic retinopathy.
